(19) United States
(12) Patent Application Publication
(10) Pub. No.: US 2026/0342948 A1
(43) Pub. Date: Oct. 29, 2026

(54) METHOD AND DEVICE FOR LASER PROJECTION

(71) Applicant: LAP GMBH LASER APPLIKATIONEN, Lüneburg (DE)

(72) Inventors: Markus Brunk, Neetze (DE); Martin Greggersen, Adendorf (DE)

(73) Assignee: LAP GMBH LASER APPLIKATIONEN, Lüneburg (DE)

(21) Appl. No.: 19/107,780

(22) PCT Filed: Aug. 1, 2023
(86) PCT No.: PCT/EP2023/071280

§ 371 Date: Feb. 28, 2025

(30) Foreign Application Priority Data
Sep. 1, 2022 (DE) 10 2022 122 155.5

(51) Int. Cl. H04N 9/31

(52) U.S. Cl. CPC H04N 9/3155; H04N 9/3129; H04N 9/3161; H04N 9/3164; H04N 9/3194

(57) ABSTRACT

A method for laser projection includes structuring a laser device to comprise a laser generation unit for generating a laser beam. An emission angle of the laser beam is adjusted by a deflection apparatus to direct the laser beam along a specified trajectory. Directing the laser beam along the specified trajectory includes establishing multiple navigation angles to be achieved by the deflection apparatus, and assigning a starting power to the navigation angles. An expected residence time of the laser beam in a navigation section is determined at least for a subset of the navigation angles (X) with which the laser beam reaches the navigation section. The respective starting power (P_{Start}) of the laser beam is adjusted to a specifiable target power depending on the determined residence time. A power of the laser beam is adjusted using a control apparatus coupled to the laser generation unit.

### CROSS-REFERENCE TO RELATED APPLICATIONS

This is a U.S. National Application of PCT/EP2023/071280, filed Aug. 1, 2023, and claiming the priority and benefit, under relevant portions of 35 U.S.C. § 119, of German Patent Application 10 2022 122 155.5, filed Sep. 1, 2022. The entire contents of said applications are hereby incorporated by reference.

### TECHNOLOGICAL FIELD

The present invention relates to a method for laser projection by means of a laser device and to a laser device which is configured to carry out the method according to the invention.

### BACKGROUND

Laser projectors are deployed in all kinds of technical fields in order to project outlines and markings onto articles. As a general rule, the lines thereof can be projected in a very visible manner onto a flat or curved surface or onto a surface of any other contour, in single-color projection, that is to say by way of example in red, green or blue projection, or in multi-color projection, in particular in mixed-color projection. CAD data, which are processed into projection data and transmitted to one or more projectors, frequently serve as the basis for the projection. Industrial laser projection systems generate virtual but visible templates on objects to be processed, which do not touch or contaminate the surfaces. Technically, the projection is usually carried out by means of one or more galvanometers, by means of which the orientation of a mirror can be changed in each case with respect to a laser beam. The laser beam is diverted by the mirror, wherein the angle of rotation of the mirror establishes the direction of the diversion. If two or more galvanometers are combined with one another, each accessible point in the space can be illuminated.

In order to comply with safety guidelines relating to the deployment and the use of lasers, it is necessary to restrict the power output by the laser projector depending on different variables such as the deployment site or intended purpose. The risk of injury to people who are in an emission region which can be reached by the laser plays a particular role. In particular, looking at powerful laser light without protection can lead to injuries to the retina and, therefore, possibly, restrictions of the eyesight.

The object of the invention is to provide a method and a device for laser projection, which allow the intensity of the laser beam to be adjusted to a specifiable level with simple means.

### SUMMARY

According to some embodiments, a method for laser projection includes using a laser device having a laser generation unit by means of which a laser beam can be generated. In some embodiments of the method, a deflection apparatus by means of which an emission angle of the laser beam is adjusted in order to direct the laser beam along a specified trajectory, and a control apparatus, coupled to the laser generation unit, by means of which the power of the laser beam can be adjusted. In some embodiments, in a first step for the navigation of the laser beam along the specified trajectory, multiple navigation angles to be achieved by means of the deflection apparatus are established, wherein a starting power is assigned in each case to the navigation angles, which starting power specifies the power of the laser beam during deflection between two navigation angles. In some embodiments, a next step depends on the number of navigation angles to be achieved directly in succession and with which the laser beam reaches a navigation section of a reference surface. An expected residence time of the laser beam in the navigation section is determined and at least for a subset of the navigation angles with which the laser beam reaches the navigation section, the respective starting power of the laser beam is in each case adjusted to a specifiable target power depending on the determined residence time.

In some embodiments, the disclosed invention makes it possible to provide a laser projector which can be operated in compliance with specified laser safety classes. For example, by utilizing the method according to the invention, a laser projector can be provided, which meets the criteria for laser class 2 and laser class 3B which can be taken from specified safety guidelines. If specific laser classes are met, it can be achieved, for example, that a laser projector according to the invention can also be deployed without a laser protection officer who is otherwise necessary for operation in the company. Compliance with specific laser classes can already be achieved by limiting the total power which can be emitted by means of the laser projection device. For example, compliance with laser class 3B can be achieved if the laser projection device does not exceed a total power of 500 mW.

Laser class 2 could likewise be achieved by means of selecting laser diodes having a maximum emitted power of 1 mW. Admittedly, the power of 1 mW is unsuitable for deployment in the projector, since the projection would not be bright enough for its intended purpose due to the usual distance from the projection surface and the high speeds at which the laser beam is usually diverted.

In order to solve this problem, a safety region can be defined, wherein specifiable criteria such as, by way of example, the criteria for laser class 2 are met outside of the safety region and other criteria, which require classification in a higher laser class, are met within the safety region. The method according to the invention is based on the idea of using safety criteria for pulsed beam sources, in particular irrespective of whether a pulsed or a non-pulsed radiation source is used for the generation of the laser beam. Consequently, for example—proceeding from currently valid safety guidelines-instead of the power, the energy of the laser beam when sweeping over an aperture of 7 mm can now be considered for compliance with the criteria necessary for a specific laser class.

The energy of the laser beam when sweeping over a specific surface section can be determined when the emitting power of the laser generation unit depending on the residence time of the laser beam within the surface section is known. In the case of a method of the type according to the invention, the trajectory to be covered by the laser beam is usually established by the sequential achievement of individual specified navigation angles by means of the deflection apparatus. The navigation angles are achieved in succession within a short interval of one another. A laser power, which establishes the laser power for said navigation angle, is usually also assigned to each navigation angle.

If the intervals between the individual achieved navigation angles are at least largely the same, the residence time of the laser beam in a specific navigation section can be determined, for example, by multiplying the number of navigation angles to be achieved in succession, with which the navigation section is reached, and the interval. For example, the interval between two navigation angles achieved in succession can be 2 μs. The interval can be specified by a time constant, which a controller uses as the process time between individual control steps.

In order to specify a curve to be projected with the laser device, it can be provided in a first step that vector graphics are provided, for example in SVG or DXF format. Said vector graphics are usually processed by means of a computer processing method set up for this purpose and broken down into individual elements such as circular sections, lines and splines. Said data are then transferred to the laser projection device. The laser projection device receives the data and processes them by means of a control device so that an angle phi, an angle theta as well as a specific laser power are assigned in each case in specifiable time periods of, by way of example, 2 μs. The solid angle to be achieved by means of the deflection apparatus, consisting of the angle phi and the angle theta, can also be referred to as the navigation angle. This processing is called trajectorization.

In some embodiments, following the trajectorization, a further step is carried out, in which method steps according to the invention are performed, in order to make possible compliance with a specific laser class. Following processing according to the invention, the data can be transferred to a controller which follows the specified trajectory cyclically and in real time with the aid of the galvanometer drives and the laser drivers based on the navigation angles. The cyclical frequencies, that is to say the repetition rates of a trajectory, are typically between 30-50 Hz.

In some embodiments, during the adjustment according to the invention of the target power of the laser beam, it is envisaged that the output power emitted by the laser generation unit is adjusted. When using a laser generation unit having multiple laser sources, the laser powers of the individual sources are preferably adjusted in order not to exceed an expected energy in a navigation section resulting from the power of the laser beam and the residence time.

In some embodiments, it is envisaged that for the adjustment of the starting powers to the target powers, an expected actual energy to be applied in the navigation section of the reference surface by means of the laser beam is determined on the basis of the specified starting powers of the laser beam and in the event of the actual energy exceeding a first specifiable limit energy, the starting powers are in each case reduced to target powers in such a way that an expected actual energy is reached which is less than or equal to the limit energy.

In some embodiments, the adjustment of the starting powers assigned to the navigation angles to the target powers can be performed in such a way that all of the starting powers to be adjusted are reduced or increased in each case by a specific factor. When using multiple laser sources in which the laser powers of the individual laser sources are assigned in each case to the navigation angles, each starting power to be adapted is reduced or increased, for example, by a specific factor.

In some embodiments of the method according to the invention, it is envisaged that the adjustment of the starting power to the target power takes place prior to the activation of the laser so that the laser does not exceed the specifiable limit power or, respectively, energy values when activated and, consequently, compliance with specific laser classes is immediately guaranteed upon activation of the laser.

In some embodiments, in addition to reducing the starting powers to the target powers, increasing the starting power also comes into consideration. An increase can be performed, by way of example, if the expected actual energy of the laser beam in a navigation section is lower than a specifiable limit energy. If the residence time of the laser beam in the navigation section does not exceed a specific level, so that a specified limit energy or, respectively, limit power of the laser beam in the considered navigation section is already fallen short of without adaptation, it can also be provided that the starting powers are left unchanged.

In some embodiments, the expected actual energy to be applied in the navigation section of the reference surface by means of the laser beam can be calculated, for example, with the aid of the equation G1.

$${E}_{ist} = {\sum }_{i = a}^{n} {t}_{i} \times {P}_{i} \times {S}_{i} \qquad ( G1 )$$

Wherein the following applies to equation G1 and subsequently: Eᵢₛₜ is the actual energy; tᵢ is the interval between a previous navigation angle and the considered navigation angle i; Pᵢ is the starting power of the laser source of the laser beam assigned to the considered navigation angle i; Sᵢ is a scaling factor of the laser source; a is the first considered navigation angle and n is the last considered navigation angle.

In equation G1, the sum is formed for a number of successive navigation angles a to n, wherein the period of time tᵢ is in each case multiplied by the laser power Pᵢ and a scaling factor Sᵢ of the laser source. If there is more than one source, the equation is to be expanded accordingly by the laser power Pₓᵢ and the scaling factor Sₓᵢ of the further sources. For example, the actual energy can be determined when using a laser generation unit having two laser sources by means of equation G2.

$${E}_{ist} = {\sum }_{i = a}^{n} ( {t}_{i} \times {P}_{1 i} \times {S}_{1 i} + {t}_{i} \times {P}_{2 i} \times {S}_{2 i} ) \qquad ( G2 )$$

In the case of laser projectors of the generic type, the period of time tᵢ between two navigation angles i is preferably the same in each case. That is to say that the period of time is, for example, formed by a time constant t_{K}, in particular a controller constant. In one application, the time constant t_{K} can be 2 μs.

The limit energy can be established for different laser power classes to be attained. For example, a limit energy for laser class 2 can be established by means of equation G3.

E_{grenz}=7×10⁻⁴×T^{0.75}×C₆ (G3)

Wherein the following applies to equation G3 and subsequently: E_{grenz} is the limit energy; T is the residence time of the laser beam in the considered navigation section; C₆ is a rating factor for the degree of focusing of the laser beam. When using a laser light which is directed in a parallel manner, that is to say a collimated laser light, in which the beam diameter is in particular 7 mm or less than 7 mm, a value of 1 can, for example, be chosen for the rating factor C₆. When using an expanded laser beam, this expansion is accordingly taken into account by the rating factor. In the case of the method according to the invention, a collimated laser beam having a beam diameter of less than 7 mm is preferably used.

The limit energy for laser class 3R can be established, for example, by means of equation G4.

E_{grenz}=3.5×10⁻³×T^{0.75} (G4)

The residence time T results from the sum of the intervals between the considered navigation angles i. If the intervals are the same in each case, that is to say are present as a time constant, the residence time T can be formed by multiplying the time constant by the number of navigation angles to be achieved directly in succession and with which the laser beam reaches the considered navigation section of the reference surface.

If the determined actual energy is higher than the limit energy to be fallen short of, it is envisaged that the starting powers of the considered navigation angles are multiplied by a correction factor in order to reduce the expected actual energy of the laser in the considered navigation section to a level that is at or below the limit energy. The correction factor can be established, for example, by means of equation G5.

$${F}_{K} = \frac{{E}_{grenz}}{{E}_{ist}} \qquad ( G5 )$$

According to a preferred embodiment, it can furthermore be provided that an expected section power to be output by means of the laser beam in a navigation section of the first reference surface is determined on the basis of the target powers of the laser beam, and wherein the section power is compared with a specifiable limit power, and wherein in the event of the expected section power exceeding the limit power, at least for a subset of the navigation angles with which the laser beam reaches the navigation section, the target powers are in each case reduced in such a way that the expected section power is less than or equal to the limit power.

In this case, it is checked whether the section power attained with specified target powers or, respectively, starting powers, inasmuch as the powers assigned to the navigation angles have not been changed, outside a specific protection region lies below a limit power to be fallen short of. If the expected section power at the distance of the protection region is greater than the limit power to be fallen short of, then the target powers of at least some of the, in particular all of the, navigation angles with which the laser beam reaches the considered navigation section are proportionally reduced.

In the case of this embodiment, it is further envisaged that for the determination of the section power, an expected section energy to be applied in the navigation section is initially determined and is linked to a repetition rate of the specified trajectory.

The section energy can be determined, for example, by utilizing equation G1, or, respectively, by using multiple laser sources by utilizing an adapted equation such as, by way of example, equation G2, wherein the actual energy corresponds to the section energy. The section power can be determined, by way of example, by utilizing equation G6, by multiplying the section energy by the repetition rate of the trajectory.

P_{Abschnitt}=E_{Abschnitt}×f_{TR} (G6)

Wherein the following applies to equation G6 and subsequently: P_{Abschnitt} is the section power; E_{Abschnitt} is the section energy and f_{TR} is the frequency or, respectively, the repetition rate of the trajectory, that is to say, for example, the number of repetitions of the trajectory which are to be projected per second.

In some embodiments, it is envisaged that for at least one navigation section of the reference surface, trajectory segments of the specified trajectory, for which the laser beam reaches said navigation section, are identified. By means of the identification of trajectory segments, with which the laser beam reaches the considered navigation section, it can initially be recognized whether more than one trajectory section lies within the considered navigation section. A total energy of different combinations of said trajectory segments can be taken into account below in order to determine whether a specifiable limit energy is exceeded. The trajectory segments are sections of the specified trajectory, which are in each case composed of directly successive navigation angles with which the laser beam reaches the considered navigation section.

In some embodiments, it is further envisaged that when more than one trajectory segment lying within the navigation section is identified for at least one combination, in particular for each combination, consisting of a first trajectory segment and one or more further directly successive trajectory segments, a number of navigation angles, assigned to the combination, of a trajectory section of the specified trajectory comprising the trajectory segments is established, beginning with a first navigation angle of the first trajectory segment of the combination and ending with a last navigation angle of the last trajectory segment of the combination along the trajectory section.

The number of possible combinations of different trajectory segments which lie in each case in the considered navigation section increases with the number of identified segments. The number of combinations can be determined utilizing equation G7. The region of the specified trajectory, which comprises identified trajectory segments of a combination, is a trajectory section assigned to the navigation section. A trajectory section is assigned to each combination of trajectory segments. A trajectory section begins with a first navigation angle of a first trajectory segment of a combination lying within the navigation section and it ends with a last navigation angle of a last trajectory segment of a combination lying within the navigation section. That is to say that such a trajectory section comprises all directly successive navigation angles beginning with a first navigation angle of a first trajectory segment and ending with a last navigation angle of a last trajectory segment of the combination.

$${N}_{K} = \frac{{n}_{TS} \times ( {n}_{TS} - 1 )}{2} \qquad ( G7 )$$

Wherein the following applies to equation G7 and subsequently: N_{K} is the possible number of combinations which results from the number n_{TS} of trajectory segments lying within the considered navigation section.

For the consideration of a combination, a first trajectory segment and one or more further directly successive trajectory segments are taken into account in each case. Directly successive trajectory segments are those segments of the trajectory which directly succeed one another in the direction of the temporal sequence of the navigation angles to be achieved or, respectively, in the direction of movement of the laser along the trajectory, that is to say, without skipping over segments lying therebetween, and likewise lie in the considered navigation section. That is to say that a combination can consist of two or more than two trajectory segments lying within the considered navigation section. It is understood that it is always only a single run-through of the trajectory within a period which is considered here. That is to say that the trajectory segments which lie within a considered navigation section are in each case different segments of a single run-through of the trajectory.

In some embodiments, it is further envisaged that a reference duration is established for at least one combination, in particular for each combination, depending on the number of navigation angles assigned to the combination, wherein a combination limit energy assigned to the combination is determined depending on the reference duration, and wherein an expected combination actual energy is determined for the combination from the sum of segment energies, which correspond in each case to the expected energy of the trajectory segments of the combination, and wherein the combination actual energy is compared with the combination limit energy, and wherein in the event of the combination actual energy exceeding the combination limit energy, at least for a subset of the navigation angles of the trajectory section, the starting power or target power of the laser beam assigned in each case to the navigation angles is reduced in such a way that the expected combination actual energy is less than or equal to the combination limit energy.

Like the limit energy E_{grenz}, the combination limit energy can also be established for different laser power classes to be attained. Similarly to equation G3, the combination limit energy can be established for laser class 2, for example, by utilizing equation G8.

E_{KGE}=7×10⁻⁴×RD^{0.75}×C₆ (G8)

Wherein the following applies to equation G8 and subsequently: E_{KGE} is the combination limit energy; RD is the reference duration for the considered combination; C₆ is the rating factor for taking into account the focusing degree of the laser beam which, as explained above, can be 1, for example.

The combination limit energy for laser class 3R can be established, for example, by similarly utilizing equation G4.

The reference duration RD can be determined from the sum of the intervals between the individual navigation angles of the considered combination, beginning with a first navigation angle of the first trajectory segment of the combination and ending with a last navigation angle of the last trajectory segment of the combination along the trajectory section. That is to say that not only are the navigation angles of the individual trajectory segments of the combination taken into account, but also those between two directly successive trajectory segments. That is to say that the reference duration RD can be established, for example, by utilizing equation G9.

$$RD = {\sum }_{i = a}^{n} {t}_{i} \qquad ( G9 )$$

Wherein the following applies to equation G9 and subsequently: RD is the reference duration for the considered combination; tᵢ is the interval between a previous navigation angle and the considered navigation angle i; a is the first considered navigation angle and n is the last considered navigation angle.

If the intervals are in each case the same between two directly successive navigation angles, that is to say that a time constant t_{K} is present, the reference duration can be determined by multiplying the time constant t_{K} by the number of considered navigation angles n, that is to say, in particular, by utilizing equation G10.

RD=n×t_{K} (G10)

The segment energies can be established by utilizing equation G11. Equation G11 can be utilized similarly to equation G1 or, respectively, when using multiple sources by expanding the equation G11 similarly to the explanations regarding equation G2. To avoid repetitions, further explanations are thus dispensed with.

$${E}_{SE} = {\sum }_{i = a}^{n} {t}_{i} \times {P}_{i} \times {S}_{i} \qquad ( G11 )$$

Wherein the following applies to equation G11 and subsequently: E_{SE} is the segment energy of a considered trajectory segment; tᵢ is the interval between a previous navigation angle and the considered navigation angle i; Pᵢ is the starting power assigned to the considered navigation angle i or, respectively, the adapted target power of the laser source of the laser beam following the utilization of the previous method steps; Sᵢ is a scaling factor of the laser source; a is the first navigation angle and n is the last navigation angle of the considered trajectory segment.

Similarly to the explanations regarding equations G1 and G2, the period of time tᵢ between two navigation angles i is preferably the same in each case for laser projectors of the generic type.

In order to adjust the power of the laser beam to a level at which the combination actual energy is less than or equal to the combination limit energy, it can be provided that the power previously assigned to the considered navigation angles is adjusted to a new power. Similarly to the preceding explanations, the laser power previously assigned to the considered navigation angles, that is to say, the starting or, respectively, target power adjusted up to this method step, can be multiplied by a factor. Therefore, the power values of the considered navigation angles are changed proportionally in each case. The power can be adjusted, for example, by utilizing equation G12.

$${P}_{neu} = {P}_{alt} \times \frac{{E}_{KGE}}{{E}_{KIE}} \qquad ( G12 )$$

Wherein the following applies to equation G12 and subsequently: Pₙₑᵤ is the new adjusted power assigned to a considered navigation angle; Pₐₗₜ is the old power assigned to a considered navigation angle; E_{KGE} is the combination limit energy; E_{KIE} is the combination actual energy.

According to a preferred embodiment, it is envisaged that the adjustment of the power of the laser beam is carried out by means of a pulse width modulation.

It can further be provided that a distance of objects detected in the detection range of the distance sensor from the laser device is monitored by means of a distance sensor. It can therefore be recognized that an object is located within a specifiable safety distance. As a matter of priority, it is envisaged that people (in particular the head of a person) who are within a safety distance are recognized by means of the distance sensor.

Primarily, it is provided that the distance sensor simply recognizes when an object is located within an adjusted safety distance. That is to say, it is not necessary to detect the actual distance. That is to say that a suitable distance sensor could be set up to capture an object located in the safety region based on a threshold value being exceeded. Alternatively or additionally, it can be provided that the distance sensor is configured and set up to determine a distance of a detected object or, respectively, to generate a signal by means of which a distance from the object can be established.

In order to increase the laser safety, it can be provided that the power of the laser beam is adjusted depending on the distance monitored by means of the distance sensor, wherein, in particular, in the event of a specifiable safety distance being fallen short of, the laser generation unit is deactivated.

In some embodiments of the invention, it can be provided that the reference surface is calculated as a virtual surface which extends over an angular range, in particular over the entire angular range, of a navigation region which can be reached by the laser beam. The navigation region which can be reached by the laser beam is substantially established based on the exit opening of the laser device of the solid angle which can be reached with the deflection apparatus.

For an equidistant distribution of the navigation sections over the projection region of the laser device, it is envisaged that the virtual reference surface has a spherical shape and extends at a distance from the laser projection device, wherein the distance of the virtual reference surface corresponds in particular to the safety distance. The distance of the virtual reference surface can be chosen, for example, according to the local conditions and/or according to specifiable safety criteria. By means of the method according to the invention, compliance with laser class 2 or compliance with another laser safety class such as, by way of example, laser class 3R is guaranteed in the distance ranges which exceed the safety distance. It is understood that the method according to the invention can also be performed with multiple different reference surfaces at different distances in order, for example, to guarantee compliance with different laser safety classes in different distance ranges.

In some embodiments, it is further envisaged that the reference surface is divided into a grid comprising multiple grid cells, wherein each grid cell corresponds to a navigation section which can be reached by the laser beam. A navigation section can, for example, have a rectangular outer contour. In particular, it can be provided that the outer contour of a navigation section is square. For the edge length of a rectangular or, respectively, square navigation section, any value can in principle be chosen, by way of example a value based on specific standardized specifications. For example, the edge length can be a value between 4 mm, or less than 4 mm, and 10 mm, or more than 10 mm. A square outer contour with an edge length of 7 mm is preferably envisaged.

In particular, in order to generate different colored laser projections, it can be provided that the laser generation unit has two or more than two laser beam sources, wherein the beams generated by means of the two or more than two laser beam sources are superimposed for the generation of the laser beam. For example, it can be provided that a red laser having a starting power assigned to the navigation angles of in each case 100% of its maximum power is used together with a green laser having a starting power assigned to the navigation angles of in each case 50% of its maximum power. A different power adjustment of the laser sources makes it possible to project different mixed colors. In the case of a power reduction according to the invention in order to comply with limit energies, it can be provided that both sources are reduced proportionally, for example the red laser to 50% and the green laser to 25% of their respective maximum power. When using multiple laser sources, the individual sources must be adapted individually, for example by means of individually adjustable pulse width modulation.

In some embodiments, it is envisaged that the deflection apparatus has at least one movable mirror, in particular at least one galvanometer mirror, by means of which the laser beam can be deflected to adjust the emission angle, for the deflection of the laser beam in different spatial directions.

A safety problem can arise if the deflection apparatus inadvertently remains non-functional or does not reach the desired deflection speed due to malfunctions. The actual residence time of the laser beam in a specific navigation section can then be higher than the calculated residence duration. In order to reduce this safety risk, it is envisaged that a specified speed of a drive unit of the deflection apparatus is monitored by means of a monitoring apparatus. During this monitoring it can be provided, for example, that it is determined whether the actual angle of the deflection apparatus corresponds to the provided target angle. Deviations between the actual angle and the target angle are also called “contouring errors.” Alternatively or additionally, it can be provided that the resulting speed from both mirror movements is checked against a limit speed. With this monitoring, mirrors which remain completely non-functional, that is to say “sticking” mirrors, can, in particular, also be recognized.

In some embodiments, if a target speed of the deflection apparatus is fallen short of, it can be provided that the laser power is reduced to a safety non-critical level. Alternatively or additionally, it can be provided that the laser device is deactivated in the event of a target speed being fallen short of.

Finally, the invention also involves a laser device for laser projection having a laser generation unit by means of which a laser beam can be generated, a deflection apparatus by means of which an emission angle of the laser beam can be adjusted in order to direct the laser beam along a specifiable trajectory, and a control apparatus, coupled to the laser generation unit, by means of which the power of the laser beam can be adjusted, wherein the laser device is configured and set up to carry out a method according to the invention in accordance with one of the previously explained embodiments.

Advantages, details and further embodiments of the laser device according to the invention are set out, in particular, by the explanations regarding the embodiments of the method according to the invention.

### BRIEF DESCRIPTION OF THE DRAWINGS

Further details and embodiments of the invention are explained with reference to the FIGURES.

FIG. 1 schematically illustrates an embodiment of a laser device according to the invention.

FIG. 2 schematically illustrates an embodiment of navigation angles to be achieved by means of some embodiments of the laser device according to the invention.

FIG. 3 schematically illustrates an embodiment of a reference surface having navigation sections.

FIG. 4 schematically illustrates an embodiment of a navigation section of the embodiment of the reference surface from FIG. 3.

FIG. 5a schematically illustrates an embodiment of a navigation section of a reference surface having a trajectory section intersecting the navigation section.

FIG. 5b schematically illustrates individual trajectory segments which lie within the navigation section from FIG. 5a.

### DETAILED DESCRIPTION

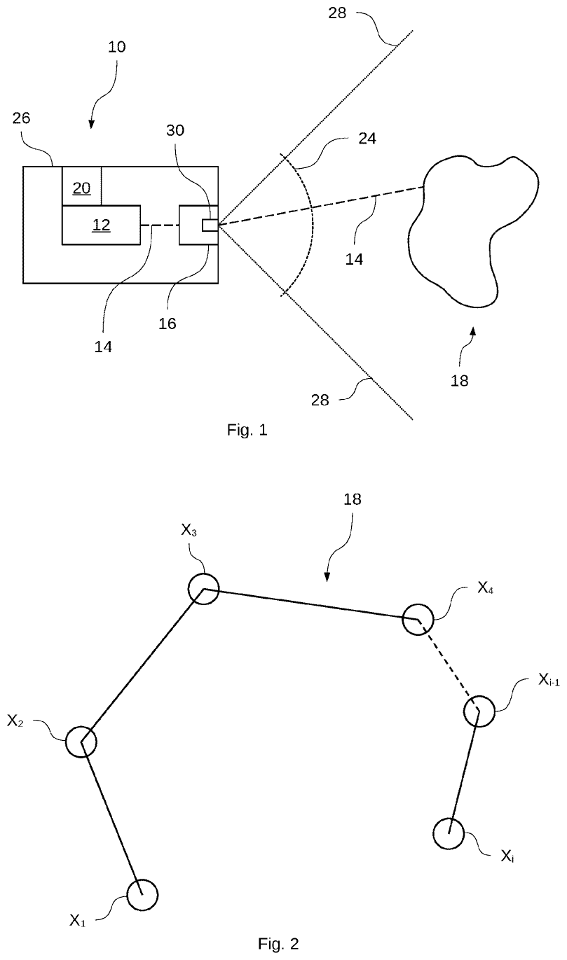

FIG. 1 shows a laser device 10, also generally referred to as a projector, having a laser generation unit 12 which generates a laser beam 14. The laser beam 14 is directed by means of a deflection apparatus 16 in such a way that the laser beam 14 covers a specifiable trajectory 18 in space. The power of the laser generation unit can be adjusted by means of a control apparatus 20 coupled to the laser generation unit 12. The control apparatus 20 and/or a further control apparatus provided for this purpose can control the deflection apparatus 16 in such a way that a specified navigation angle X (FIG. 2) is achieved. A distance sensor 30 can be provided to capture objects in a detection range of the distance sensor 30. The deflection apparatus 16 can be set up to direct the laser beam 14 into any navigation angle X located in a navigation region 28. The navigation region 28 can be configured to be conical in the emission region of the laser device 10. The detection range of the distance sensor 30 is preferably larger or at least as large as the navigation region 28 which can be reached with the laser beam 14.

As indicated schematically in FIG. 1, the laser generation apparatus 12, the control apparatus 20 and the deflection apparatus 16 can be arranged in a common housing 26. The distance sensor can likewise be arranged inside, but also on or outside the housing.

In the navigation region 28 of the laser device 10, a reference surface 24 is configured—here shaped spherically to the deflection point of the laser beam 14 on the deflection apparatus 16. Said reference surface 24 can in particular be arranged at a safety distance which corresponds to a safety region monitored by the distance sensor 30. The reference surface 24 is a virtually generated spatial surface which serves to adjust, according to the invention, the power of the laser device 10.

In a specific example which can be used as the basis for any embodiment of the invention, it can be provided in a first criterion 1 that the power of the laser sources of the laser device is adjusted by two mechanisms. By adjusting a diode current of a laser diode on the laser driver, the maximum laser power which is to be emitted can be specified. Said value is preferably adjusted once and is preferably unchanging. On the other hand, a largely linear adjustment of the emitted power can be realized by a pulse width modulation of the signal at the laser diode. Said intensity value, which can be adjusted by means of a power value, is established for each point of the trajectory and is used in order, e.g., to make the projected figure appear equally bright. The power adjustment can be influenced in order to avoid any violation of the laser class. The basic frequency of said pulse width modulation is, for example, 20 MHz and therefore has a period of 50 ns which, in turn, can be divided into 271 stages of 184 μs each. Said pulses therefore have a minimum length of 184 μs and a maximum length of 50 ns. Consequently, they fall under this criterion 1.

FIG. 2 shows, purely schematically, a simple example of a spatial trajectory 18 to be achieved with the laser beam 14 of a laser device 10. The trajectory 18 is formed by achieving multiple navigation angles X. The navigation angles X₁ to Xᵢ depicted in FIG. 2 for illustrative purposes are achieved in succession by means of the deflection apparatus 16. Since the navigation angles X are in each case achieved in succession at an interval of, for example, 2 μs, the route covered by the laser beam 14 between two navigation angles X is, ideally, a straight line.

The navigation angles X are formed from two items of angle information phi and theta. With this pair of angles phi and theta, any solid angles within the navigation region 28 of the laser device 10 can be achieved. The items of angle information phi and theta serve to adjust the deflection apparatus 16. A power adjustment of the laser generation unit 12, which establishes the intensity of the laser beam 14 during the control of the laser beam 14 between two navigation angles X, is assigned to each navigation angle X.

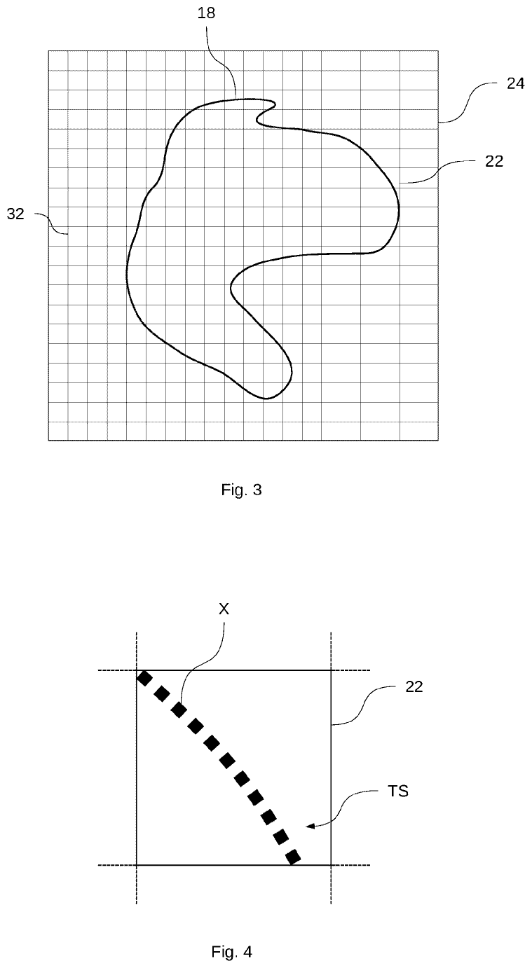

FIG. 3 shows a virtual reference surface 24 in a schematic diagram. The reference surface 24 is divided into a grid having grid cells 32 which have a square configuration in each case in the present example, wherein each grid cell 32 corresponds to a navigation section 22 which can be reached by the laser beam 14. FIG. 3 also shows, purely schematically, a trajectory 18 covered by the laser beam 14. As depicted, the laser beam 14 reaches different navigation sections 22 when achieving the navigation angles X of the trajectory 18.

FIG. 4 shows a navigation section 22 from FIG. 3 in detail. In this schematic detailed representation, successively achieved navigation angles X of a segment of the trajectory 18 lying within the navigation section 22 are shown. The region of the trajectory 18 which lies within the navigation section 22 is referred to as the trajectory segment TS. In this schematic diagram, eleven navigation angles X lie within the navigation section 22. If an interval between the navigation angles X is constant, the residence time of the laser beam 14 within the considered navigation section 22 can be calculated by multiplying the time constant and the number of navigation angles of the partial segment TS. For example, if the time constant is 2 μs, the resulting residence time is 22 μs (11×2 μs).

In a specific example which can be used as the basis for any embodiment of the invention, it can be provided in a second criterion 2, as already explained above, that a trajectorized projection 18 in the projection region 28 is distributed on a grid 32 of a reference surface 24. Mathematically, this grid 32 preferably lies at the distance of the safety region from the projector 10 and more preferably has a spherical shape. The entire region 28 which can theoretically be reached by the projector 10 is covered by the sphere. The preferably square cells of the grid 32 of the sphere preferably have an edge length of 7 mm in both directions on the spherical surface. Each point of the trajectory 18 is assigned to a cell or, respectively, a navigation section 22 of the grid or, respectively, the reference surface 24 based on its coordinates or, respectively, navigation angles X.

Regarding each navigation section 22 of the reference surface 24 it is known when the laser beam 14 enters the navigation section 22 and exits again. The part between the entry and exit of a cell constitutes a pulse or, respectively, a trajectory segment TS. If the same navigation section 22 is hit again at a later time of the trajectory 18, this is judged to be a further trajectory segment TS.

FIG. 4 schematically shows, for example, an individual cell or, respectively, an individual navigation section 22 from the example in FIG. 3. The subregion of the trajectory 18 is symbolized by points representing navigation angles X which, viewed in terms of time, each represent a subregion having a constant period of time of, by way of example, 2 μs each of the trajectory 18.

For criterion 2, the energy is established for each 2 μs step, and indeed from the known maximum laser power of the sources and the adjustable power by the pulse width modulation. The sum over all 2 μs steps in this pulse is the pulse energy. The permissible limit value is produced, for example, by utilizing equation G3 with the time which is established by the number of pulses multiplied by 2 μs.

With this example, compliance with laser class 2 can be made possible, for example, in a specific safety region. Similarly to the procedure for establishing laser class 2 at the distance of the safety region, compliance with laser class 3R can be made possible for the distance of, for example, 100 mm in front of the projector. In both cases, a grid having an edge length of 7 mm can be used, which leads to different pulse lengths of the trajectory segments TS and power adjustment values for the considered navigation angles X. In both cases, in the event that the calculated limit values are exceeded, the power values for the navigation angles X of the pulses, or respectively, trajectory segments TS considered in each case are corrected, so that this is below the limit energy.

In a further specific example which can be used as the basis for any embodiment of the invention, it can be provided in a third criterion 3 that the power adjustments of the navigation angles X corrected, by way of example, after taking criterion 2 into account, are re-evaluated. According to this criterion 3, the sum of the energies for all pulses or, respectively, trajectory segments TS, which occur in a grid cell or, respectively, in a navigation section 22 within a specific time of, for example, 0.25 seconds, is considered. This also includes the repetitions of the pulses or, respectively, trajectory segments TS which occur within the considered navigation section 22 due to the projection frequency of, for example, 25 to 50 Hz. In the case of this criterion 3 it can be provided that the total power of a cell or, respectively, of a navigation section 22 may not be larger than 1 mW at the distance of the protection region and not more than 5 mW at a distance of 100 mm (cf. laser class 2 or 3R, respectively). If the limit is exceeded, a correction is performed for the entire cell, that is to say, for all of the navigation angles X with which the laser beam 14 reaches the navigation section 22. For this purpose, all of the power values of the navigation angles X of the pulses or, respectively, trajectory segments TS of the cell or, respectively, of the navigation section 22 are reduced proportionally, so that the value is below the limit.

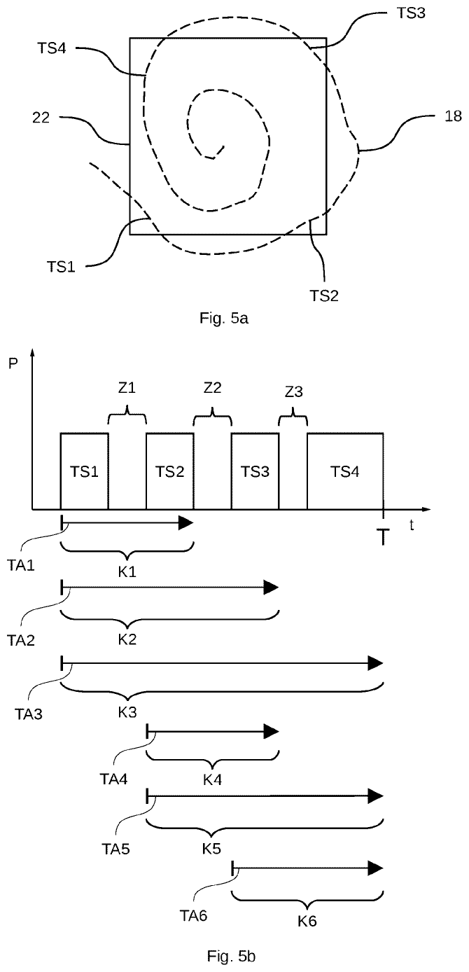

FIG. 5a shows a schematic diagram of a further example of a navigation section 22 having a section of a trajectory 18, wherein four subsegments TS1 to TS4 lie within the navigation section.

FIG. 5b schematically shows the compilation of various combinations of subsegments TS1 to TS4 from FIG. 5a lying within the navigation section 22. Between the subsegments TS1 to TS4, intermediate pieces ZI to Z3 are indicated, which represent the trajectory regions which lie along the trajectory 18 between the first trajectory segment TS1 reaching the navigation section 22 in time and the last trajectory segment TS4 reaching the navigation section 22 and therefore lie outside the navigation section 22.

In the present case, in one run-through of the trajectory 18, four trajectory segments TS lie within the navigation region 22. According to equation GL7, six combinations K1 to K6 can be formed, which are formed in each case from two or more than two directly successive trajectory segments TS. In the present case, the combination K1 consists of the trajectory segments TS1 and TS2. Combination K2 consists of the trajectory segments TS1, TS2 and TS3. The remaining combinations K3 to K6 are formed accordingly. Each combination K defines a trajectory section TA which comprises the navigation angles X of the trajectory 18, beginning with a first navigation angle X of the first trajectory segment TS of the considered combination K and ending with the last navigation angle X of the last trajectory segment TS of the considered combination K. In the present case, the trajectory section TA1 comprises, for example, all of the navigation angles X of the trajectory 18 beginning with the first navigation angle X of the first trajectory segment TS1 of the considered combination K1 and ending with the last navigation angle X of the last trajectory segment TS2 of the considered combination K1. The remaining trajectory sections TA2 to TA6 are defined accordingly.

In a specific example which can be used as the basis for any embodiment of the invention, it can be provided in a fourth criterion 4 that multiple pulses, that is to say, multiple trajectory segments TS occurring in a navigation section 22 within a period T are to be considered. This occurs, for example, when the trajectory 18 intersects itself. An example of a cell or, respectively, of a navigation section 22 having multiple trajectory segments TS within a period T is—as mentioned-depicted in FIG. 5a. In FIG. 5b, the temporal course of the four trajectory segments TS1 to TS4 within this cell or, respectively, the navigation section 22 is indicated schematically. All four trajectory segments TS are run through within a period T of a trajectory 18. This can be seen in the temporal sequence in the diagram in FIG. 5b. The diagram shows, purely schematically, the power P of the trajectory segments TS over time. The navigation angles X of the trajectory segment TS1 are initially achieved, then the trajectory segments TS2 to TS4 follow. Then the period T is ended and a new run-through begins.

The four individual pulses TS1 to TS4 can already be taken into account in criterion 2. Admittedly, since the pulses can occur irregularly in number, length and distance within a periodic cycle, further virtual pulses, also referred to as trajectory sections TA, must be checked against the limits from criterion 2. This can be carried out for different laser classes, that is to say, for example both for laser class 2 and for laser class 3R. The virtual pulses or, respectively, trajectory sections TA are defined by considering multiple successive pulses together. The successive pulses or, respectively, trajectory segments TS of a virtual pulse are also referred to here as combination K. Specifically, this can be understood by looking at FIG. 5b. In this example cell having four pulses TS1 to TS4, six additional virtual pulses or, respectively, trajectory sections TA1 to TA6 are defined. Depending on the power class considered, equations G3 and G4 from criterion 2 can, for example, be utilized by means of the power and the duration of each trajectory section TA in order to determine the limit value for the respective virtual pulse or, respectively, the respective trajectory section TA. The virtual pulses or, respectively, trajectory sections TA1 to TA6 are in each case depicted in FIG. 5b as an arrow under the diagram. For the first virtual pulse TA1, the sum of the energies of the first two individual pulses TS1 and TS2 of the combination K1 is established as well as the length, that is to say, the reference duration RD, of the trajectory section TA1 from the beginning of the trajectory segment TS1 up to the end of the trajectory segment TS2. For the second virtual pulse TA2, the sum of the energies of the first three individual pulses TS1 to TS3 is established as well as the length, that is to say, the reference duration RD, of the trajectory section TA2 from the beginning of the trajectory segment TS1 up to the end of the trajectory segment TS3. The further virtual pulses TA3 to TA6 are considered accordingly.

### LIST OF REFERENCE NUMERALS

10 Laser device
12 Laser generation unit
14 Laser beam
16 Deflection apparatus
18 Trajectory
20 Control apparatus
22 Navigation section
24 Reference surface
26 Housing
28 Navigation region
30 Distance sensor
32 Grid
X Navigation angle
TS Trajectory segment
K Combination
Z Intermediate pieces
TA Trajectory section

1-17. (canceled)

18. A method for laser projection, comprising:
structuring a laser device to comprise a laser generation unit;
generating a laser beam by means of the laser generation unit;
adjusting an emission angle of the laser beam by means of a deflection apparatus in order to direct the laser beam along a specified trajectory, wherein directing the laser beam along the specified trajectory comprises,
(i) establishing multiple navigation angles (Xi) to be achieved by means of the deflection apparatus,
(ii) assigning a starting power in each case to the navigation angles (X), wherein the starting power specifies the power of the laser beam during deflection between two navigation angles (X),
(iii) determining an expected residence time of the laser beam in a navigation section and at least for a subset of the navigation angles (X) with which the laser beam reaches the navigation section, and
(iv) adjusting the respective starting power (P_{Start}) of the laser beam in each case to a specifiable target power depending on the determined residence time; and
adjusting a power of the laser beam by means of a control apparatus coupled to the laser generation unit.

19. The method according to claim 18, wherein adjusting the respective starting power (P_{Start}) of the laser beam in each case to a specifiable target power comprises,
determining an expected actual energy (Eᵢₛₜ) to be applied in the navigation section of the reference surface by the laser beam based on the specified starting powers (P_{Start}) of the laser beam (14), and
determining when an actual energy (Eᵢₛₜ) exceeds a first specifiable limit energy (E_{grenz}), and in response, reduce the starting powers (P_{Start}) to target powers (P_{Soll}) in such that an expected actual energy (Eᵢₛₜ) is reached and is less than or equal to a limit energy (E_{grenz}).

20. The method according to claim 18, further comprising:
determining an expected section power (P_{Abschnitt}) to be output in a navigation section of a first reference surface by the laser beam based on the target powers (P_{Soll}) of the laser beam,
comparing the section power with a specifiable limit power (P_{grenz}), and
reducing the target powers (P_{Soll}) such that the expected section power (P Abschnitt) is less than or equal to the limit power (P_{grenz}) when the expected section power exceeds the limit power (P_{grenz}), at least for a subset of the navigation angles (Xi) with which the laser beam reaches the navigation section.

21. The method according to claim 20, wherein the determining of the expected section power comprises determining an expected section energy to be applied in the navigation section, and linking the expected section energy to a repetition rate of the specified trajectory.

22. The method according to claim 18, further comprising identifying trajectory segments (TS) of the specified trajectory for which the laser beam reaches the navigation section for at least one navigation section of the reference surface.

23. The method according to claim 22, wherein when more than one trajectory segment (TS) lying within the navigation section is identified for at least one combination (K) comprising a first trajectory segment (TS) and one or more further directly successive trajectory segments (TS), a number of navigation angles (X) assigned to the combination (K) of a trajectory section (TA) of the specified trajectory comprising the trajectory segments (TS) is established beginning with a first navigation angle (X) of the first trajectory segment (TS) of the combination (K) and ending with a last navigation angle (X) of the last trajectory segment (TS) of the combination (K) along the trajectory section (TA).

24. The method according to claim 23, further comprising:
determining a reference duration (RD) for the at least one combination (K) depending on the number of navigation angles (X) assigned to the combination (K);
determining a combination limit energy (E_{KGE}) assigned to the combination (K) depending on the reference duration (RD);
determining an expected combination actual energy (E_{KIE}) for the combination (K) from a sum of segment energies (E_{SE}), which corresponds to an expected energy of the trajectory segments (TS) of the combination (K);
comparing the combination actual energy (E_{KIE}) with the combination limit energy (E_{KGE}); and
reducing the starting power or target power of the laser beam assigned to the navigation angles (X) such that the expected combination actual energy (E_{KIE}) is less than or equal to the combination limit energy (E_{KGE}) when the combination actual energy (E_{KIE}) exceeds the combination limit energy (E_{KGE}), at least for a subset of the navigation angles (X) of the trajectory section (TA).

25. The method according to claim 18, wherein the adjustment of the power of the laser beam is carried out by means of a pulse width modulation.

26. The method according to claim 18, further comprising monitoring a distance of objects detected in a detection range of a distance sensor from the laser device using a distance sensor.

27. The method according to claim 26, further comprising adjusting the power of the laser beam based on the distance detected by the distance sensor, and deactivating the laser generation unit when the distance detected is less than a specifiable safety distance.

28. The method according to claim 18, further comprising calculating the reference surface as a virtual surface which extends over an angular range of a navigation region which can be reached by the laser beam.

29. The method according to claim 28, wherein a virtual reference surface comprises a spherical shape and extends at a distance from the laser projection device, and wherein the distance of the virtual reference surface corresponds to a safety distance.

30. The method according to claim 18, further comprising dividing the reference surface into a grid comprising multiple grid cells, wherein each grid cell corresponds to a navigation section which can be reached by the laser beam.

31. The method according to claim 18, wherein the laser generation unit comprises two or more laser beam sources, wherein the beams generated by the two or more laser beam sources are superimposed for the generation of the laser beam.

32. The method according to claim 18, wherein the deflection apparatus comprises at least one movable mirror configured to deflect the laser beam to adjust the emission angle.

33. The method according to claim 18, further comprising monitoring a specified speed of a drive unit of the deflection apparatus by means of a monitoring apparatus.

34. A laser device for laser projection configured to carry out a method according to claim 18, comprising:
a laser generation unit (12) configured to generate a laser beam (14) can be generated;
a deflection apparatus (16) configured to adjust an emission angle of the laser beam (14) to direct the laser beam (14) along a specifiable trajectory (18); and
a control apparatus coupled to the laser generation unit and configured to adjust a power of the laser beam (14).
apparatus (20), coupled to the laser generation unit (12), by means of which the power of the laser beam (14) can be adjusted, wherein the laser device (10) is configured and set up to carry out a method according to any one of claims 1 to 16.
